(19) United States
(12) Patent Application Publication
(10) Pub. No.: US 2026/0263406 A1
(43) Pub. Date: Sep. 10, 2026

(54) SYNERGISTIC COMPOSITION FOR MODULATING EXPRESSION OF CD38

(71) Applicant: CELAGENEX RESEARCH (INDIA) PVT. LTD., Thane, Maharashtra (IN)

(72) Inventors: Rajaram SAMANT, Thane (IN); Manoj TONGRA, Thane (IN)

(21) Appl. No.: 19/491,380

(22) PCT Filed: Dec. 20, 2023
(86) PCT No.: PCT/IN2023/051199

§ 371 Date: Dec. 8, 2025

(30) Foreign Application Priority Data
Jun. 9, 2023 (IN) 202321039610

(51) Int. Cl. A61K 31/202; A61K 31/155; A61K 31/194; A61K 31/197; A61K 31/20; A61K 31/22; A61K 31/575; A61K 38/05

(52) U.S. Cl. CPC A61K 31/202; A61K 31/155; A61K 31/194; A61K 31/197; A61K 31/20; A61K 31/22; A61K 31/575; A61K 38/05

(57) ABSTRACT

The invention disclosed herein relates to synergistic composition for modulating expression of cyclic ADP ribose hydrolase (CD38). Particularly, the present invention relates to synergistic bioactive composition comprising therapeutic blend of bioactive sesquiterpenoid and at least one potent glucose uptake promoters present in specific weigh percentage along with pharmaceutically acceptable excipients. The present synergistic composition is useful in improving intracellular signaling activity, particularly the composition is used for treating upregulated cyclic ADP ribose hydrolase (CD38) disorders.

### TECHNICAL FIELD OF THE INVENTION

The present invention relates to a synergistic composition for treating diseases or disorders related to upregulation cyclic ADP ribose hydrolase (CD38). Particularly the present invention relates a synergistic bioactive composition wherein the composition comprising exogenous combination of bioactive sesquiterpenoid and at least one glucose uptake promoter along with pharmaceutically acceptable excipients. The present synergistic composition is useful in improving intracellular signaling activity, particularly the composition is used for treating upregulated cyclic ADP ribose hydrolase (CD38) disorders.

### BACKGROUND OF THE INVENTION

CD38 (cluster of differentiation 38), also known as cyclic ADP ribose hydrolase is a glycoprotein found on the surface of many immune cells. CD38 is a molecule that can act as an enzyme, with NAD-depleting and intracellular signaling activity, or as a receptor with adhesive functions. CD38 is a single chain glycoprotein with a single transmembrane segment and can topologically behave as a type II or type III membrane protein depending on its membrane orientation. CD38 is a transmembrane glycoprotein that functions as an enzyme, specifically as an ADP-ribosyl cyclase. It is found in various cell types, including immune cells, and is involved in multiple physiological processes, such as calcium signaling, cell adhesion, and immune regulation. Particularly, CD38 is expressed at high levels on multiple myeloma (MM) cells and at relatively low levels on normal lymphoid and myeloid cells and some non-hematopoietic tissue [Wang et al. J Exp Clin Cancer Res (2022) 41:210].

CD38 catalyses the conversion of NAD⁺ to cyclic ADP-ribose (cADPR), a calcium mobilizing messenger molecule. It is primarily known for its role in regulating intracellular calcium levels and cell signaling pathways. Dysregulation of CD38 leads to different kind of diseases such as cancer, tumor growth, metastasis, immune evasion, neurodegenerative disorders such as Parkinson's disease and multiple sclerosis, brain and spinal cord disorders, such as sleep disorders, depression, pain and Alzheimer derived memory impairments.

Specifically, administration of bioflavonoid, a CD38 inhibitor, to obese mice increases NAD+ levels and improves glucose and lipid homeostasis [Nutrients. 2021 November; 13 (11): 3734]. Further, bioflavonoid has predominant role in controlling blood glucose level along with the protection of vital organs eventually damaged during diabetes, by minimizing toxicities and associated diabetic complications [American Journal of Pharmacology and Toxicology 9 (1): 39-52, 2014]. The beneficial outcomes of CD38 absence or inhibition seem to be a consequence of enhanced energy expenditure, and this effect is mediated at least in part via a NAD+-dependent activation of SIRT-PGC1α axis, involved in the regulation of mitochondrial biogenesis and energy homeostasis [FASEB J. 2007; 21:3629-3639].

U.S. Pat. No. 9,845,482 B2 discloses CD38 inhibitor and the NAD precursor are present in the medium in an amount effective to increase the number of functional mitochondria in the mammalian oocyte, oogonial stem cell (OSC).

WO2022/170265 A1 discloses a method of treating, inhibiting, decreasing, reducing, ameliorating and/or preventing inflammatory injury to a donor organ or tissue in a recipient subject comprising administering to the recipient subject an effective amount of a small molecule CD38 inhibitor.

Adipose tissue plays an important role in glucose homeostasis and affects insulin sensitivity in other tissues. In obesity and type 2 diabetes, glucose uptake promoter 4 (GLUT4) is downregulated in adipose tissue, and glucose transport is also impaired in muscle. It is observed that overexpression of GLUT4 selectively in adipose tissue could prevent insulin resistance when glucose transport is impaired in muscle. Various biochemical changes in GLUT4 and CD38 protein expression patterns and histopathological alterations in some vital organs such as liver, kidneys and pancreas were investigated to compare the antidiabetic potentials of these two chemicals and to understand their capability to control the damages of the vital organs during diabetes or other metabolic disorder. Regulation of CD38 expression and NAD (P) content in adipose tissue during thermogenesis or during inflammation have been studied. CD38 expression is regulated in opposite directions during thermogenesis or during inflammation. In thermogenesis, CD38 downregulation is paralleled by an increase in NAD+ levels in BAT, and by an increase in NADPH in WAT.

CD38 protein plays a significant role in cellular health, where it is prominent marker of cellular activation expressed by plasma cells, T cells, NK cells and other hematopoietic cell types during various stages of maturation. It has been connected to HIV infection, leukemias, myelomas, PCOS, solid tumors, type II diabetes mellitus and bone metabolism, as well as some genetically determined conditions.

The present invention aims to address the need of the art for effectively modulating expression of CD38 to treat metabolic disorders and provides for a bioavailable composition where the combination of bioactive sesquiterpenoid with at least one glucose uptake promoter; along with pharmaceutically acceptable excipients afford significant alteration or modulation in upregulated CD38 expression in a subject in need thereof.

### OBJECTIVE OF THE INVENTION

The primary objective of the invention is to provide composition that modulate expression of CD38 to treat metabolic disorders.

Another objective of the invention is to provide synergistic combination of bioactive compounds present in specific weight concentration to regulate glucose homeostasis.

Yet another objective of the invention is to provide synergistic combination of bioactive sesquiterpenoid along with at least one glucose uptake promoter present in specific weight concentration to regulate glucose homeostasis.

Further, another objective of the invention is to provide cost effective, non-toxic composition of a biomolecule for human administration that facilitates transportation of bioactive molecule in a therapeutically effective amount for the treatment of CD38 upregulated metabolic disorders.

### SUMMARY OF THE INVENTION

To meet the above objectives, the inventors of the present invention carried out thorough experiments to establish significant therapeutic effects of the active ingredients or biomolecules or biological material or peptides or terpenoid or amino acids, bioactive promoter or nutrients present in the composition for improving metabolic function in a subject in need thereof in safer way.

In an aspect, the present invention relates to synergistic compositions comprising therapeutically active biomolecule along with pharmaceutically acceptable carriers for treating metabolic neurological, age-related disorders.

In yet another aspect, the present invention provides synergistic bioactive composition comprising bioactive sesquiterpenoid in combination potent glucose uptake promoters that modulates intracellular CD38 expression.

In another aspect, the present invention provides synergistic combination of bioactive sesquiterpenoid and at least one potent glucose uptake promoters, wherein the bioactive sesquiterpenoid is (2Z,4E)-5-[(1S)-1-hydroxy-2,6,6-trimethyl-4-oxocyclohex-2-en-1-yl]-3-methylpenta-2,4-dienoic acid; and potent glucose uptake promoter is selected from the group consisting of Oxobutanedioic acid; 2-Oxo-1,5-pentanedioic acid; (3β,5β,7α,12α)-7,12-dihydroxy-3-[(1-oxoeicosyl)amino]-cholan-24-oic acid; (2R)-3-(Allylthio)-2-aminopropanoic acid; 1-(4-Aminobutyl)guanidine; dileucine; (3R)-3-acetyloxy-4-(trimethylazaniumyl)butanoate; 3-amino-2-methylpropanoic acid; (2S)-2-[[(2S)-2hydroxypropanoyl]amino]-3-phenylpropanoic acid, Pentadecanoic acid, (2R,3S,4S)-pentane-1,2,3,4,5-pentol, 1-isothiocyanato-4-methylsulfinylbutane, 3-Aminoisobutyric acid, (2R,4aS,6aR,6aS,14aS,14bR)-10-hydroxy-2,4a,6a,6a,9,14a-hexamethyl-11-oxo-1,3,4,5,6,13, 14,14b-octahydropicene-2-carboxylic acid or salts thereof present in specific weight concentration along with pharmaceutically acceptable excipients.

In still another aspect, the present invention discloses synergistic bioactive composition for improving intracellular signaling activity, particularly the composition is used for treating upregulated cyclic ADP ribose hydrolase (CD38) disorders such as weight gain, diabetes, obesity, cancer, liver diseases, sexual dysfunction, insulin resistance, hyperglycemia, glucose intolerance, type 1 and 2 diabetes, prediabetes, inflammation, polycystic ovary syndrome (PCOS), neurodegenerative diseases, cardiovascular diseases, osteoarthritis, musculoskeletal diseases, neuromuscular diseases, and kidney diseases.

### ABBREVIATIONS

GI: Gastrointestinal
WAT: White Adipose Tissue
BAT: Brown Adipose Tissue
NADPH: Nicotinamide adenine dinucleotide phosphate

### BRIEF DESCRIPTION OF FIGURES

FIG. 1 illustrates band intensity of GLUT4 expression in percentage at 54 kDa

FIG. 2 illustrates band intensity of CD38 expression in percentage at 45 kDa

FIG. 3 illustrates % increase in GLUT4 level as compared to disease control

FIG. 4 illustrates % decrease in CD38 level as compared to disease control

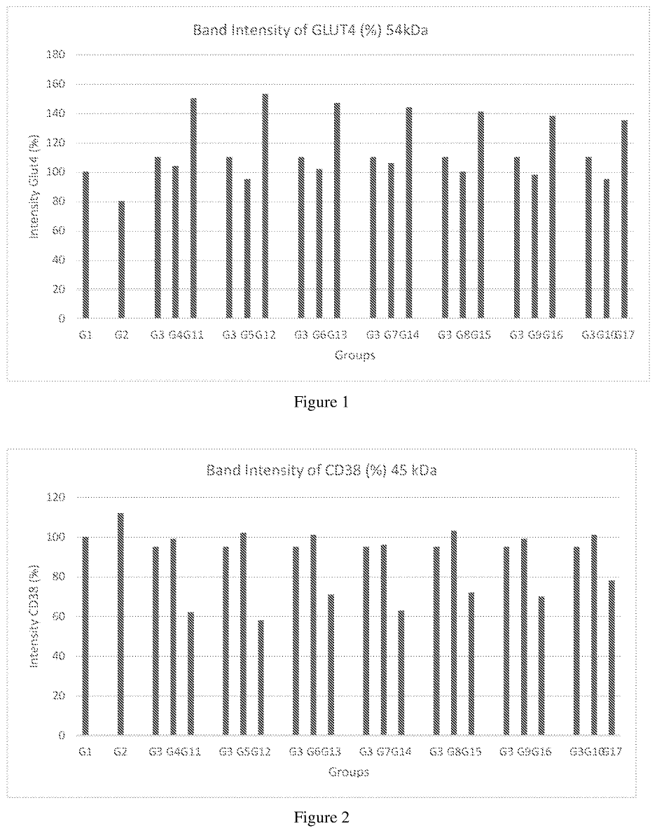

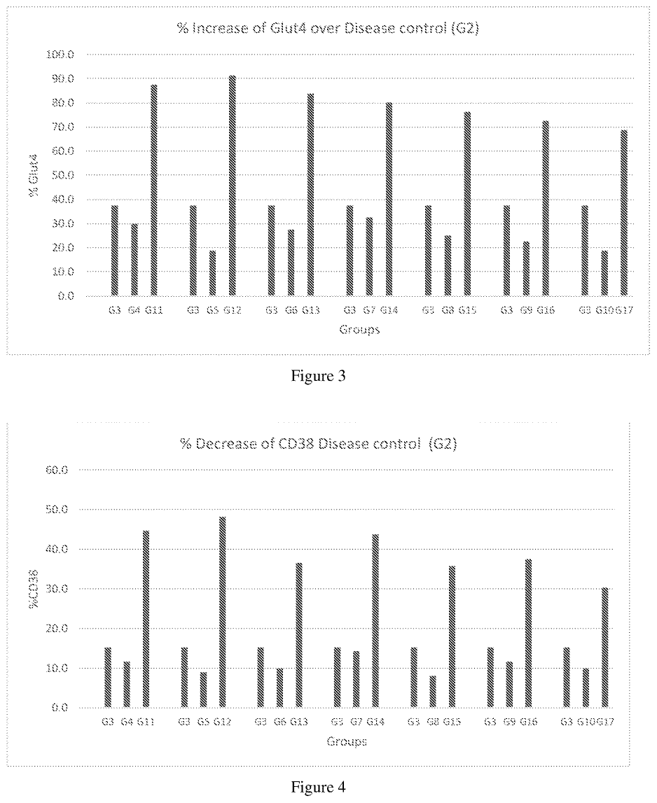

### DETAILED DESCRIPTION OF THE INVENTION

The present invention will now be described in detail in connection with certain preferred and optional embodiments, so that various aspects thereof may be more fully interpreted and comprehended. However, any skilled person or artisan will appreciate the extent to which such embodiments could be generalized in practice. It is further to be understood that all terminology used herein is for the purpose of describing embodiments only and is not intended to be limiting in any manner or scope.

Unless defined otherwise, all technical and scientific expressions used herein have the same meaning as commonly understood by one of ordinary skill in the art to which embodiments of the invention pertains.

In describing and claiming the embodiments of the present invention, the following terminology will be used in accordance with the definitions set out below which are known in the state of art.

The singular forms “a,” “an,” and “the” include plural reference unless the context clearly dictates otherwise. Also, the term ‘composition’ does not limit the scope of the invention for multiple compositions that can be illustrated for best mode of the invention.

The term “pharmaceutically/nutraceutically acceptable salt,” as used herein, represents those salts which are within the scope of sound medical judgment, suitable for use in contact with the tissues of humans and animals without undue toxicity, irritation, allergic response and the like and are commensurate with a reasonable benefit/risk ratio.

Particularly, the term “pharmaceutically acceptable salts” refers to the relatively non-toxic, inorganic, and organic acid addition salts of compounds, amino acid salt, sugar-based salt, alkali, or alkaline earth metal salts, as well as solvates, co-crystals, polymorphs, and the like of the salts.

The term “therapeutically active” is an ingredient which is accountable for a therapeutic effect in human.

All modifications and substitutions that come within the meaning of the description and the range of their legal equivalents are to be embraced within their scope. A description using the transition “comprising” allows the inclusion of other elements to be within the scope of the invention.

The term “sesquiterpenoid” pertains to a group of 15 carbon compounds derived by the assembly of 3 isoprenoid units and they are found mainly in higher plants but also in invertebrates. Sesquiterpene structures present in acyclic, mono-, bi-, tri-, and tetracyclic systems.

The term ‘exogenous’ refers to any material that is present and active in an individual organism or living cell but that originated and prepared outside that organism for the purpose of administration.

In a preferred embodiment, the present invention relates to a synergistic composition for modulating intracellular expression of CD38.

In a preferred embodiment, the present invention provides a synergistic bioactive composition comprising therapeutic blend of bioactive sesquiterpenoid with at least one potent glucose uptake promoters along with pharmaceutically acceptable excipients for treating CD38 upregulated metabolic disorders.

In a preferred embodiment, the present invention provides a synergistic bioactive composition of (2Z,4E)-5-[(1S)-1-hydroxy-2,6,6-trimethyl-4-oxocyclohex-2-en-1-yl]-3-methylpenta-2,4 dienoic acid with at least one potent glucose uptake promoters along with pharmaceutically acceptable excipients.

In a particular embodiment, the present synergistic composition comprises at least one potent glucose uptake promoters, wherein the potent glucose uptake promoter is selected from the group consisting of Oxobutanedioic acid; 2-Oxo-1,5-pentanedioic acid; (3B,5B,7a, 12a)-7,12-dihydroxy-3-[(1-oxoeicosyl)amino]-cholan-24-oic acid; (2R)-3-(Allylthio)-2-aminopropanoic acid; 1-(4-Aminobutyl) guanidine; dileucine; N-(2-Hydroxyethyl) hexadecanamide; (3R)-3-acetyloxy-4-(trimethylazaniumyl) butanoate;3-amino-2-methylpropanoic acid; (2S)-2-[[(2S)-2-hydroxypropanoyl]amino]-3-phenylpropanoic acid or salts thereof present in specific weight ratio along with pharmaceutically acceptable excipients.

In another embodiment, the present invention provides synergistic combination of bioactive sesquiterpenoid with potent glucose uptake promoter; wherein the sesquiterpenoid is (2Z,4E)-5-[(1S)-1-hydroxy-2,6,6-trimethyl-4-oxocyclohex-2-en-1-yl]-3-methylpenta-2,4 dienoic acid; wherein potent glucose uptake promoters are selected from group consisting of Oxobutanedioic acid; 2-Oxo-1,5-pentanedioic acid; (3B,5B,7a, 12a)-7,12-dihydroxy-3-[(1-oxoeicosyl)amino]-cholan-24-oic acid; (2R)-3-(Allylthio)-2-aminopropanoic acid; 1-(4-Aminobutyl) guanidine; dileucine; (3R)-3-acetyloxy-4-(trimethylazaniumyl) butanoate; 3-amino-2-methylpropanoic acid; (2S)-2-[[(2S)-2hydroxypropanoyl]amino]-3-phenylpropanoic acid, Pentadecanoic acid, (2R,3S,4S)-pentane-1,2,3,4,5-pentol, 1-isothiocyanato-4-methylsulfinylbutane, 3-Aminoisobutyric acid, (2R,4aS,6aR,6aS,14aS,14bR)-10-hydroxy-2,4a,6a,6a,9,14a-hexamethyl-11-oxo-1,3,4,5,6,13, 14,14b-octahydropicene-2-carboxylic acid or salts thereof present in specific weight concentration along with pharmaceutically acceptable excipients.

In yet another preferred embodiment, the present invention provides synergistic bioactive composition comprising therapeutically effective amount of bioactive sesquiterpenoid, wherein the sesquiterpenoid is present in the range of 0.001% to 1% by weight of the total composition, preferably 0.002% to 0.75% by weight of total composition.

In yet another preferred embodiment, the present invention provides synergistic bioactive composition comprising therapeutically effective amount of potent glucose uptake promoters; wherein the glucose uptake promoter is present in the range of 10% to 99% by weight of the total composition.

In yet another embodiment, the present synergistic composition regulates glucose homeostasis, where it affects insulin secretion and sensitivity in pancreatic beta cells.

Further, the composition helps to modulate blood glucose levels and potentially have implications for diabetes management.

In another embodiment, the present synergistic composition inhibits the production of pro-inflammatory cytokines and enzymes, potentially exerting a protective effect against inflammation-related disorders.

Particularly, the present composition is useful in glucose homeostasis, inflammation modulation, and immune regulation.

In another embodiment, the present synergistic composition inhibits oxidative stress and increasing energy expenditure via activating NAD+/Sirtuins signaling pathways in muscle and brown fat.

CD38 regulates glucagon-induced glucose production and gluconeogenesis related gene expression in primary hepatocytes HCs.

Glucose Uptake promoter Type 4 (GLUT4) is an insulin-regulated membrane protein responsible for decreasing blood glucose concentration. Thus, facilitation of GLUT4 translocation by modulation of insulin signaling pathway and modulation of CD38 protein can be potential therapeutic target for the treatment of diabetes.

In yet another embodiment the present composition improves glucose transport and oxidation, mitochondrial biogenesis, insulin sensitivity and promotes glucose uptake.

In another embodiment, the present invention provides synergistic pathway for modulating CD38 and GLU4 expression. The composition ingredient act as a potent agonist for lanthionine synthetase C-like 2 receptor and peroxisome proliferator-activated receptors family member (PPARs).

Further the composition stimulates Ca2+ release by activation of downstream targets including phospholipase C/protein kinase C (PLC-PKC) cascade and adenylate cyclase CAMP-dependent protein kinase A (PKA) pathway.

In another preferred embodiment the present composition exhibits novel nutritional therapeutic for controlling glucose metabolism and that regulate glucose load in both genetically and dietary-induced subject.

In another embodiment, the composition of present invention is useful for improving glucose homeostasis and metabolic diseases such as but not limiting to obesity and obesity related diseases, inflammation, glucose intolerance, PCOS and stress.

In yet another embodiment the present composition is also useful in memory and cognitive processes, particularly it diminishes cognitive deficiency in Alzheimer's disease subjects.

In yet another embodiment, the present invention provides an synergistic bioactive composition comprising therapeutically effective amount of bioactive sesquiterpenoid, wherein the sesquiterpenoid is present in the range of 0.1-1000000 mcg of total composition, preferably, in the range of 0.1-10000 mcg. (1000 mcg equal to 1 mg).

The present synergistic composition has predominant role in controlling blood glucose level along with the protection of vital organs eventually damaged during diabetes, by reducing toxicities and associated diabetic complications which act as a potential antidiabetic agent.

In yet another embodiment, the present invention provides synergistic bioactive composition comprising therapeutically effective amount of potent glucose uptake promoters or salts thereof present in the range of 0.1-1000 mg of total composition, preferably, in the range of 1-700 mg.

In yet another embodiment, the present invention provides synergistic bioactive composition comprising therapeutically effective amount of potent glucose uptake promoters selected from group comprising Oxobutanedioic acid; 2-Oxo-1,5-pentanedioic acid; (3β,5β,7α,12α)-7,12-dihydroxy-3-[(1-oxoeicosyl)amino]-cholan-24-oic acid; (2R)-3-(Allylthio)-2-aminopropanoic acid; 1-(4-Aminobutyl) guanidine; dileucine; (3R)-3-acetyloxy-4-(trimethylazaniumyl) butanoate; 3-amino-2-methylpropanoic acid; (2S)-2-[[(2S)-2hydroxypropanoyl]amino]-3-phenylpropanoic acid, Pentadecanoic acid, (2R,3S,4S)-pentane-1,2,3,4,5-pentol, 1-isothiocyanato-4-methylsulfinylbutane, 3-Aminoisobutyric acid, (2R,4aS,6aR,6aS,14aS,14bR)-10-hydroxy-2,4a,6a,6a,9,14a-hexamethyl-11-oxo-1,3,4,5,6,13, 14,14b-octahydropicene-2-carboxylic acid or salts thereof.

The term therapeutic blend or exogenous blend indicates the combination of active ingredients in specific concentration excluding the excipients.

The term “therapeutically effective amount” denotes an amount that reduces the risk, potential, possibility or occurrence of a disease or disorder, or provides advanced alleviation, mitigation, and/or reduction or restoration or modulation, regulation of at least one indicator/biomarker (e.g., blood or serum CRP level), and/or minimize at least one clinical symptom related to CD38 upregulated metabolic disorder like PCOS, diabetes, etc.

The term “subject in need thereof” pertains to subject preferably mammal, more preferably human suffering or suspected with metabolic disorders like PCOS, diabetes, etc.

In the context of the present invention, the term “treatment” relates to alleviate, mitigate, prevent, prophylaxis, attenuate, manage, regulate, modulate, control, minimize, lessen, decrease, down regulate, up regulate, moderate, inhibit, restore, suppress, reverse, limit, block, decrease, stabilize, ameliorate, or cure, heal metabolic disorders like PCOS, diabetes, etc.

Notably, the instant synergistic composition is non-hazardous, non-toxic, active ingredient and safe for human consumption without any adverse effects, therefore the present composition can also be used under preventive therapy/adjuvant therapy/add-on therapy/combination/adjunctive therapy in a subject in need thereof.

Certain compounds of the present invention can exist in unsolvated forms as well as solvated forms, including hydrated forms. Further some compounds of the present invention can exist in multiple crystalline or amorphous forms (“polymorphs”). Compounds of the present invention can be formulated in geometric or, enantiomeric or stereoisomeric forms.

As used herein, the term “pharmaceutically acceptable carriers, diluents or excipients” is purported to mean, without limitation, any adjuvant, carrier, excipient, sweetening agent, diluents, preservative, dye/colorant, flavour enhancer, surfactant, wetting agent, dispersing agent, suspending agent, stabilizer, isotonic agent, solvent, emulsifier, or encapsulating agent, encapsulating polymeric delivery systems or polyethylene glycol matrix, which is acceptable for use in the subject, preferably humans.

Excipients may also include, for example: antiadherents, antioxidants, binders, coatings, compression aids, disintegrants, dyes (colors), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, or waters of hydration, salts.

In another embodiment, the present invention relates to synergistic nutritional composition, which can be prepared in a manner well known in the pharmaceutical art, and can be administered by a variety of routes, depending upon whether local or systemic treatment is desired and upon the area to be treated. The preferable route of administration includes but not limited to sublingual, rectal, topical, parenteral, nasal, or oral.

In some embodiment, the instant synergistic-composition can be administered to the subject in need thereof, in the form which is suitable for oral use, such as a tablet, capsule (in the form of delayed release, extended release, sustained release, enteric coated release); hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray. Further the composition can be formulated for parenteral use including intravenous, subcutaneous, intramuscular, intravascular, infusion, intraperitoneal, intracerebral, intracerebroventricular, or intradermal.

The magnitude of a prophylactic or therapeutic dose typically varies with the nature and severity of the condition to be treated and the route of administration. The dose, and perhaps the dose frequency, will also vary according to the age, body weight and response of the individual patient.

In another embodiment, the total daily dose (in single or divided doses) ranges from about 1 mg per day to about 5000 mg per day, preferably about 10 mg per day to about 1500 mg per day.

The present invention also relates to a method of modulating expression of CD38 to treat metabolic disorders, said method comprising the step of administering the combination or composition of the present invention to a subject in need thereof.

The present invention also relates to a method of improving intracellular signaling activity of CD38, wherein the method comprising the step of administering the combination or composition of the present invention to a subject in need thereof.

In yet another embodiment, the present composition economically viable effect on insulin release and stimulates glucose uptake by brown adipose tissue and browning gene expression and mitochondrial biogenesis in beige preadipocytes in the white adipose tissue.

Similar to brown adipocytes, beige adipocytes are rich in mitochondria that express uncoupling protein 1 (UCP1) and can accomplish thermogenesis. Notably, beige adipocytes primarily contribute to energy expenditure rather than to energy storage.

The composition further controls body weight homeostasis and the white adipose tissue-to-brown adipose tissue ratio.

In some embodiments, the upregulated CD38 metabolic disorder treated by the present or composition include but is not limited to weight gain, obesity, adiposity, dyslipidemia, hypertension, cancer, liver diseases, sexual dysfunction, insulin resistance, hyperglycemia, glucose intolerance, type 1 and 2 diabetes, prediabetes prediabetes, inflammation, polycystic ovary syndrome (PCOS), neurodegenerative diseases, cardiovascular diseases, osteoarthritis, musculoskeletal diseases, neuromuscular diseases, neuropathic pain, and kidney diseases or any combination thereof.

In another embodiment, the synergistic-composition of the present invention is non-toxic, cost effective, enriched with bioactive ingredients, and provides safeguard against problems associated with CD38 upregulated metabolic dysfunction without any adverse effect.

In another preferred embodiment, the present synergistic bioactive composition downregulates CD38 expression by 30% to 48% and upregulates GLUT4 expression by 68% to 91% when compared with disease control.

The term ‘subject in need thereof’ pertains to a human, preferably diabetic, overweight, male or females of reproductive age. Particularly female including but not limited to adult, women planning for pregnancy, women before conception, pregnant women, conceive, lactating women, women of reproductive age.

Compound or pharmaceutically acceptable salts includes, hydrates, halides like chloride, bromide, metal salts like calcium, sodium, potassium, hydroxide, phosphate; polymorphs, solvates, enantiomers or racemates. Some of the crystalline forms of the compound exist as polymorphs and as such are intended to be included in the present disclosure. In addition, some of the compounds may form solvates with water (i.e., hydrates) or common organic solvents, and such solvates are intended to be encompassed by some embodiments.

In one of the embodiments, the present invention provides a synergistic bioactive composition that is present in effective amount along with pharmaceutically acceptable excipients.

As used herein, the term “pharmaceutically acceptable carriers, diluents or excipients” is purported to mean, without limitation, any adjuvant, carrier, excipient, sweetening agent, diluents, preservative, dye/colorant, flavor enhancer, surfactant, wetting agent, dispersing agent, suspending agent, stabilizer, isotonic agent, solvent, emulsifier, or encapsulating agent, encapsulating polymeric delivery systems or polyethylene glycol matrix which is acceptable for use in the subject, preferably humans. Excipients may also include, for example: anti-adherents, antioxidants, binders, coatings, compression aids, disintegrants, dyes (colors), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, buffering agents or waters of hydration, salts.

In another embodiment, the present invention relates to synergistic bioactive composition, which can be prepared in a manner well known in the pharmaceutical art, and can be administered by a variety of routes, depending upon whether local or systemic treatment is desired and upon the area to be treated. The preferable route of administration includes but not limited to sublingual, rectal, topical, parenteral, nasal, or oral.

In some embodiment, the present synergistic-composition can be administered to the subject in need thereof, in the form which is suitable for oral use, such as a tablet, capsule (in the form of delayed release, extended release, sustained release, enteric coated release); hard gelatin capsules, hard gel, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray. Further, the composition can be formulated for parenteral use including intravenous, subcutaneous, intramuscular, intravascular, infusion, intraperitoneal, intracerebral, intracerebroventricular, or intradermal.

Synergistic bioactive composition of the present invention is suitable for oral administration and can be presented as discrete units such as capsules (e.g., soft-gel capsules), cachets or tablets each containing a predetermined amount of the active ingredient; as a powder or granules; as a solution or a suspension in an aqueous liquid or a non-aqueous liquid, syrup; or as an oil-in-water liquid emulsion or a water-in-oil liquid emulsion. The active ingredients can also be presented in the form of a bolus, electuary or paste, bioactive bar, energy bars (candy bars), powder, energy drink, ready to drink, granule sachet.

Further, the present composition can be formulated in the form of age-appropriate pediatric oral dosage forms such as syrup, minitablets, chewable formulations, orodispersible films, orodispersible tablets and bioadhesive buccal tablets. It can also be prepared in the form of snack, confectionery food products, sachet, gummies.

In another embodiment of the present invention, the diluents are selected from starches, hydrolyzed starches, partially pregelatinized starches, anhydrous lactose, cellulose powder, lactose monohydrate, sugar alcohols such as sorbitol, xylitol and mannitol, silicified microcrystalline cellulose, ammonium alginate, calcium carbonate, calcium lactate, dibasic calcium phosphate (anhydrous/dibasic dehydrate/tribasic), calcium silicate, calcium sulphate, cellulose acetate, corn starch, pregelatinized starch, dextrin, β-cyclodextrin, methylated-β-cyclodextrin, dextrates, dextrose, erythritol, ethyl cellulose, fructose, fumaric acid, glyceryl palmitostearate, magnesium carbonate, magnesium oxide, maltodextrin, maltose, medium chain triglycerides, polydextrose, polymethacrylates, sodium alginate, sodium chloride, sterilizable maize, sucrose, sugar spheres, talc, trehalose, xylitol, vehicles like petrolatum, dimethyl sulfoxide and mineral oil or the like.

In some embodiment of the present invention, the diluent in the composition/formulation is present in a range of 1% to 30% by weight of the total composition/formulation.

In yet another embodiment of the present invention, the binder is selected from disaccharides such as sucrose, lactose, polysaccharides and their derivatives like starches, cellulose, or modified cellulose such as microcrystalline cellulose and cellulose ethers such as hydroxypropyl cellulose (HPC); hydroxypropyl methyl cellulose (HPMC); sugar alcohols suchas xylitol, sorbitol, or mannitol; protein like gelatin; synthetic polymers such as polyvinylpyrrolidone (PVP), polyethylene glycol (PEG), starch, acacia, agar, alginic acid, calcium carbonate, calcium lactate, carbomers, carboxymethylcellulose sodium, carrageenan, cellulose acetate phthalate, chitosan, copovidone, corn starch, pregelatinized starch, cottonseed oil, dextrates, dextrin, dextrose, ethyl cellulose, guar gum, hydrogenated vegetable oil, mineral oil, hydroxyethyl cellulose, hydroxymethyl cellulose, hydroxyl ethyl methyl cellulose, hydroxypropyl cellulose, inulin, cellulose, methyl cellulose, polyvinylpyrrolidone and polyethylene glycol, lactose, liquid glucose, hypromellose, magnesium aluminium silicate, maltodextrin, maltose, methyl-cellulose, microcrystalline cellulose, pectin, poloxamer, polydextrose, polymethacrylates, povidone, sodium alginate, stearic acid, sucrose, sunflower oil, various animal vegetable oils, and white soft paraffin, paraffin, flavorants, colorants and wax.

In a further embodiment of the present invention, the binder in the composition/formulation is present in a range of 0.1 to 30% by weight of the composition/formulation.

In some embodiment, the antioxidant is selected from tocopherol (vitamin E), sesamol, guaiac resin, methionine, beta-carotene, lycopene, lutein, zeaxanthin, butylated hydroxy anisole (BHA), butylated hydroxytoluene (BHT), sodium ascorbate, sodium metabisulfite (SMB), 1-carnosine, propyl gallate (PG), tertiary butyl hydroquinone, cysteine (CYS), citric acid, tartaric acid, phosphoric acid, and ascorbic acid.

In another embodiment of the present invention, the amount of antioxidant in the composition/formulation is present in the range of 0.1 to 10% by wt. of the composition/formulation.

In another embodiment of the present invention, the lubricant is selected from magnesium stearate, zinc stearate, calcium stearate, glycerin monostearate, glyceryl behenate, glyceryl palmitostearate, hydrogenated castor oil, hydrogenated vegetable oil, light mineral oil, magnesium lauryl sulphate, medium-chain triglycerides, mineral oil, myristic acid, palmitic acid, poloxamer, polyethylene glycol, sodium benzoate, sodium chloride, sodium lauryl sulphate, sodium stearyl fumarate, stearic acid, talc, potassium, or sodium benzoate or the like.

In another embodiment of the present invention, the lubricant in the composition/formulation is present in a range of 0.1% to 10.0% by weight of the total composition/formulation.

In another embodiment of the present invention, the solubilizing agent is selected from polysorbate 80, sodium lauryl sulphate, anionic emulsifying wax, nonionic emulsifying wax, glyceryl monooleate, phospholipids, polyoxyethylene alkyl ethers, polyoxyethylene castor oil derivatives, polyoxyethylene sorbitan fatty acid esters, polyoxyethylene stearates, polyoxylglycerides, sorbitan esters, triethyl citrate, vitamin E, polyethylene glycol succinate, microcrystalline cellulose, carboxymethylcellulose sodium, diethanolamine, ethylene glycol palmitostearate, glycerin monostearate, hypromellose, hypromellose, acetate succinate, lecithin, polyethylene alkyl ethers, aluminum oxide, poly(methylvinyl ether/maleic anhydride), calcium carbonate, crospovidone, cyclodextrins, fructose, hydroxpropyl betadex, oleyl alcohol, povidone, benzalkonium chloride, benzethonium chloride, be 5 nzyl alcohol, benzyl benzoate, cetylpyridinium chloride, inulin, meglumine, poloxamer, pyrrolidone, sodium bicarbonate, starch, stearic acid, sulfobutylether beta cyclodextrin, tricaprylin, triolein, docusate sodium, glycine, alcohol, self-emulsifying glyceryl monooleate, cationic benzethonium chloride, cetrimide, xanthan gum, lauric acid, myristyl alcohol, butylparaben, ethylparaben, methylparaben, propylparaben, sorbic acid or the like.

In one of the embodiments of the present invention, the glidant is selected from colloidal silicon dioxide, magnesium stearate, fumed silica (colloidal silicon dioxide), starch, talc, calcium phosphate tribasic, cellulose powdered, hydrophobic colloidal silica, magnesium oxide, zinc stearate, magnesium silicate, magnesium trisilicate, silicon dioxide or the like.

In another embodiment of the present invention, the glidant in the composition/formulation is present in a range of 0.1% to 5.0% by weight of the total composition/formulation.

In some embodiment of the present invention, the stabilizers are selected from the group consisting of alginate, agar, carrageen, gelatin, guar gum, gum arabic, locust bean gum, pectin, starch, xanthan gum, trehalose and likewise.

In some embodiment of the present invention, the stabilizer in the composition/formulation is present in a range of 0.1% to 10.0% by weight of the total composition/formulation.

In some embodiment of the present invention, the plasticizers added to coating of the formulation are selected from the group consisting of propylene glycol, glycerol, glyceryl triacetate (triacetin), triethyl citrate, acetyl triethyl citrate, diethyl phthalate, acetylated monoglycerides, castor oil, mineral oil and like thereof.

In some embodiment of the present invention, the plasticizer in the composition/formulation is present in a range of 0.1% to 5.0% by weight of the total composition/formulation.

In some embodiment of the present invention, the solvent is selected from water, alcohol, isopropyl alcohol, propylene glycol, mineral oil, benzyl alcohol, benzyl benzoate, flavored glycol, carbon dioxide, castor oil, corn oil (maize), cottonseed oil, dimethyl ether, albumin, dimethylacetamide, ethyl acetate, ethyl lactate, medium-5 chain triglycerides, methyl lactate, olive oil, peanut oil, polyethylene glycol, polyoxyl, castor oil, propylene carbonate, pyrrolidone, safflower oil, sesame oil, soybean oil, sunflower oil, water-miscible solvents, organic polar or non-polar solvents or mixtures thereof.

In a preferred embodiment of the present invention, the solvent in the composition/formulation is used in a quantity sufficient to make the weight of the composition/formulation 100% by weight.

The additional additives include a polymer, a plasticizer, a sweetener, and a powdered flavor, a preservative, a colorant, a surfactant, and other excipients. The powdered flavor composition includes a flavourant associated with a solid carrier. Coating materials such as synthetic polymers, shellac, corn protein (zein) or other polysaccharides, gelatin, fatty acids, waxes, shellac, plastics, and plant fibers and like thereof are used.

In a preferred embodiment of the present invention, the additives are used in a range of 0.1 to 10% w/w of unit dose.

In yet another embodiment, the present invention provides the composition/formulation comprising a therapeutic blend of (2Z,4E)-5-[(1S)-1-hydroxy-2,6,6-trimethyl-4-oxocyclohex-2-en-1-yl]-3-methylpenta-2,4 dienoic acid with at least one potent glucose uptake promoters along with pharmaceutically acceptable excipients, wherein the pharmaceutical excipients are selected from a diluent, a binder, a lubricant, a glidant, an additive, a surfactant, a stabilizer or mixtures thereof.

In a preferred embodiment, the present invention provides the composition/formulation wherein the pharmaceutically acceptable excipients are selected from a group consisting of the diluent is present in a range of 1 to 30%; the binder present is present in a range of 0.1 to 25%; the lubricant is present in a range of 0.1 to 10.0%; the glidant is present in a range of 0.1 to 5.0%; the additive is present in a range of 0.1 to 10%; the surfactant is present in a range of 0.1 to 5.0%; the stabilizer is present in a range of 0.1 to 5.0%; %; the antioxidant is present in a range of 0.1 to 5.0%; and the plasticizer is present in a range of 0.1 to 5.0%; by weight of total composition.

In some optional embodiment of the present invention, the buffering or alkalizing agent in the composition/formulation is present in a range of 20% to 50% by weight of the total composition/formulation.

In some embodiment of the present invention, the buffering agent or alkalizing agent is selected from acetate, phosphate, citrate, and glutamate wherein the citrate salt is selected from sodium citrate, potassium citrate, monosodium citrate, citric acid anhydrous.

In further embodiment, compositions containing compounds of the present invention can be conveniently presented in unit dosage form and prepared by any of the methods well known in the art of pharmacy. Preferred unit dosage formulations are those containing an effective dose, or an appropriate fraction thereof, of the active ingredient, or a pharmaceutically acceptable salt thereof.

The magnitude of a prophylactic or therapeutic dose typically varies with the nature and severity of the condition to be treated and the route of administration. The dose, and perhaps the dose frequency, will also vary according to the age, body weight and response of the individual patient.

In general, the total daily dose (in single or divided doses) ranges from about 1 mg per day to about 2500 mg per day, preferably about 10 mg per day to about 1500 mg per day.

In certain embodiments, the present invention provides the synergistic bioactive composition wherein the effective unit dose for an oral administration is formulated in solid form which is present in a range of 5 to 1500 mg, preferably 10 to 1000 mg.

It is further recommended that women planning for pregnancy, initially receive low doses and that the dosage be titrated based on individual physiological responses and/or pharmacokinetics. It can be necessary to use dosages outside these ranges in some cases, as will be apparent to those in the art.

The present composition can be used as infant formula as well as adult formula by varying the concentration of active ingredients. Further, it is noted that the dietician or nutritionist or certified physician, medical practitioner knows how and when to interrupt, adjust, or terminate therapy in conjunction with an individual patient's response.

The use of any and all examples, or exemplary language (e.g., such as) provided herein, is intended merely to better illuminate the invention, and does not pose a limitation on the scope of the invention unless otherwise claimed.

Various other examples of compositions and modifications or adaptations thereof can be devised by a person skilled in the art after reading the foregoing preferred embodiments without departing from the spirit and scope of the invention. All such further examples, modifications and adaptations are included within the scope of the invention.

It will be appreciated by those versed in the art that the present invention makes available novel and useful-compositions, which have effects in several administration forms. Also, it will be understood by those with knowledge in the dietary supplement and nutraceutical art, that many embodiments of this invention may be made without departing from the spirit and scope of the invention, and the invention is not to be construed as limited, as it embraces all equivalents therein.

The invention may be further illustrated by the following examples, which are for illustrative purposes only and should not be construed as limiting the scope of the invention in anyway.

The present disclosure is therefore to be considered as in all respects illustrative and not restrictive, the scope of the invention being indicated by the appended claims and examples, and all changes or alterations which come within the ambit of equivalency are intended to be encompassed therein.

### EXAMPLES

Having described the basic aspects of the present invention, the following non-limiting examples illustrate specific embodiments thereof. Those skilled in the art will appreciate that many modifications may be made in the invention without changing the essence of invention.

### Example 1: Various Compositions/Formulations

i. Composition 1: Tablet/Capsule

| Ingredient | mg per unit dose |
|---|---|
| (2Z,4E)-5-[(1S)-1-hydroxy-2,6,6-trimethyl- | 100 mcg |
| 4-oxocyclohex-2-en-1-yl]-3- | |
| methylpenta-2,4-dienoic acid | |
| 3-Aminoisobutyric acid | 250 mg |
| Magnesium Stearate | 0.1-10 |
| Hydroxypropyl Methylcellulose | 0.5-10 |
| Microcrystalline Cellulose | 0.1-10 |
| Polyvinylpyrrolidone | 0.1-10 |
| Starch | 0.1-5 |
| Talc | 0.1-5 |
| Mannitol | 0.1-5 |
| Propylene Glycol | QS |
| Water | QS |
| Average weight | 260-300 mg |

ii. Composition 2: Tablet/Capsule

| Ingredient | mg per unit dose |
|---|---|
| (2Z,4E)-5-[(1S)-1-hydroxy-2,6,6-trimethyl- | 100 mcg |
| 4-oxocyclohex-2-en-1-yl]-3- | |
| methylpenta-2,4-dienoic acid | |
| 1-isothiocyanato-4-methylsulfinylbutane | 35 mg |
| Magnesium Stearate | 0.1-5 |
| Ascorbic acid | 0.1-5 |
| Microcrystalline Cellulose | 0.1-10 |
| Colloidal Silicon dioxide | 0.1-5 |
| Hydroxypropyl Methylcellulose | 0.1-10 |
| PVPP | 0.1-5 |
| Zinc Stearate | 0.1-5 |
| Talc | 0.1-5 |
| Tween 80 | 0.1-5 |
| Mannitol | 0.1-5 |
| Alcohol | QS |
| Water | QS |
| Average weight | 40-70 mg |

iii. Composition 3: Tablet/Capsule

| Ingredient | mg per unit dose |
|---|---|
| (2Z,4E)-5-[(1S)-1-hydroxy-2,6,6-trimethyl- | 100 mcg |
| 4-oxocyclohex-2-en-1-yl]-3- | |
| methylpenta-2,4-dienoic acid | |
| Oxobutanedioic acid | 100 mg |
| Microcrystalline Cellulose | 0.1-5 |
| Silicon dioxide | 0.1-5 |
| Glycerin | 0.1-5 |
| Ethyl Cellulose | 0.1-2 |
| Hydroxypropyl Methylcellulose | 0.1-10 |
| Magnesium Stearate | 0.1-5 |
| Polyvinylpolypyrrolidone | 0.1-10 |
| Talc | 0.1-5 |
| Polysorbate 20 | 0.1-2 |
| Mannitol | 0.1-2 |
| IPA | QS |
| Water | QS |
| Average weight | 110-150 mg |

iv. Composition 4: Tablet/Capsule

| Ingredient | mg per unit dose |
|---|---|
| (2Z,4E)-5-[(1S)-1-hydroxy-2,6,6- | 100 mcg |
| trimethyl-4-oxocyclohex-2-en- | |
| 1-yl]-3-methylpenta-2,4-dienoic acid | |
| (2S)-2-[[(2S)-2-hydroxypropanoyl] | 35 mg |
| amino]-3-phenylpropanoic acid | |
| Microcrystalline Cellulose | 0.1-10 |
| Colloidal silicon dioxide | 0.1-5 |
| Hydroxypropyl Methylcellulose | 0.1-5 |
| Magnesium Stearate | 0.1-5 |
| Polyvinylpyrrolidone | 0.1-5 |
| Calcium Phosphate | 0.1-5 |
| Ascorbic Acid | 0.1-1 |
| Polysorbate 20 | 0.1-2 |
| Talc | 0.1-5 |
| Sucrose | 0.1-1 |
| Mannitol | 0.1-1 |
| Glycerol | 0.1-2 |
| Average weight | 40-60 mg |

v. Composition 5: Tablet/Capsule

| Ingredient | mg per unit dose |
|---|---|
| (2Z,4E)-5-[(1S)-1-hydroxy-2,6,6- | 100 mcg |
| trimethyl-4-oxocyclohex-2-en-1- | |
| yl]-3-methylpenta-2,4-dienoic acid | |
| Pentadecanoic acid | 250 mg |
| Microcrystalline Cellulose | 0.1-5 |
| Silicon dioxide | 0.1-5 |
| Hydroxypropyl Methylcellulose | 0.1-10 |
| Magnesium Stearate | 0.1-2 |
| Zinc Stearate | 0.1-5 |
| Polyvinylpyrrolidone | 0.1-5 |
| Mineral Oil | 0.1-2.5 |
| Sodium benzoate | 0.1-1 |
| Ascorbic Acid | 0.1-2 |
| Polysorbate 20 | 0.1-1 |
| Talc | 0.1-5 |
| Dextrose | 0.1-1 |
| Mannitol | 0.1-1 |
| Water | QS |
| Average weight | 255-300 mg |

vi. Composition 6: Tablet/Capsule

| Ingredient | mg per unit dose |
|---|---|
| (2Z,4E)-5-[(1S)-1-hydroxy-2,6,6- | 100 mcg |
| trimethyl-4-oxocyclohex-2-en-1- | |
| yl]-3-methylpenta-2,4-dienoic acid | |
| Dileucine | 500 mg |
| Silicon Dioxide | 0.1-2 |
| Medium-chain triglycerides | 0.1-5 |
| Microcrystalline Cellulose | 0.1-10 |
| Magnesium Stearate | 0.1-5 |
| Polyvinylpyrrolidone | 0.1-5 |
| Talc | 0.1-5 |
| Corn Starch | 0.1-5 |
| Sodium ascorbate | 0.1-2 |
| Propylene glycol | 0.1-1 |
| Water | QS |
| Average weight | 510-560 mg |

vii. Composition 7: Tablet/Capsule

| Ingredient | mg per unit dose |
|---|---|
| (2Z,4E)-5-[(1S)-1-hydroxy-2,6,6- | 100 mcg |
| trimethyl-4-oxocyclohex-2-en- | |
| 1-yl]-3-methylpenta-2,4-dienoic acid | |
| ((2R,4aS,6aR,6aS,14aS,14bR)-10- | 10 mg |
| hydroxy-2,4a,6a,6a,9,14a-hexamethyl- | |
| 11-oxo-1,3,4,5,6,13,14,14b- | |
| octahydropicene-2-carboxylic acid | |
| Microcrystalline Cellulose | 0.1-5 |
| Silicon dioxide | 0.1-5 |
| Hydroxypropyl Methylcellulose | 0.1-5 |
| Magnesium Stearate | 0.1-5 |
| Zinc Stearate | 0.1-5 |
| Polyvinylpyrrolidone | 0.1-5 |
| Mineral Oil | 0.1-2 |
| Sodium benzoate | 0.1-1 |
| Ascorbic Acid | 0.1-5 |
| Polysorbate 20 | 0.1-1 |
| Talc | 0.1-5 |
| Mannitol | 0.1-1 |
| Water | QS |
| Average weight | 12-20 mg |

viii. Composition 8: Tablet/Capsule

| Ingredient | mg per unit dose |
|---|---|
| (2Z,4E)-5-[(1S)-1-hydroxy-2,6,6- | 100 mcg |
| trimethyl-4-oxocyclohex-2-en- | |
| 1-yl]-3-methylpenta-2,4-dienoic acid | |
| 1-isothiocyanato-4-methylsulfinylbutane | 35 mg |
| Potassium Citrate | 25 mg |
| Magnesium Stearate | 0.1-5 |
| Ascorbic acid | 0.1-5 |
| Microcrystalline Cellulose | 0.1-10 |
| Colloidal Silicon dioxide | 0.1-5 |
| Hydroxypropyl Methylcellulose | 0.1-5 |
| PVPP | 0.1-5 |
| Talc | 0.1-5 |
| Tween 80 | 0.1-5 |
| Mannitol | 0.1-1 |
| Alcohol | QS |
| Water | QS |
| Average weight | 65-100 mg |

ix. Composition 9: Tablet/Capsule

| Ingredient | mg per unit dose |
|---|---|
| (2Z,4E)-5-[(1S)-1-hydroxy-2,6,6- | 100 mcg |
| trimethyl-4-oxocyclohex-2-en- | |
| 1-yl]-3-methylpenta-2,4-dienoic acid | |
| 1-isothiocyanato-4-methylsulfinylbutane | 35 mg |
| 3-Aminoisobutyric acid | 250 mg |
| Microcrystalline Cellulose | 0.1-10 |
| Silicon dioxide | 0.1-5 |
| Hydroxypropyl Methylcellulose | 0.1-5 |
| Stearic acid | 0.1-5 |
| Pregelatinized starch | 0.1-10 |
| Talc | 0.1-5 |
| Tween 80 | 0.1-5 |
| Polydextrose | 0.1-5 |
| PEG | QS |
| Water | QS |
| Average weight | 290-350 mg |

### Example 2

“To evaluate the antidiabetic potential of the present compositions and its mechanistic role in controlling damages of vital tissues in streptozotocin-induced diabetic rats.”

### Test System and Animal Husbandry

Species: Rats
Strain: Sprague-Dawley
Sex: Male
No. of animals: 36 Animals (n=6 per group)
Body weight: 110-130 g
   Animal House conditions
Lighting: 12/12-hour light-dark cycle
Temperature: 22±3° C.
Relative Humidity: 40 to 60%

Animals had continuous access to fresh, potable, uncontaminated drinking water.
Feed: Normal chow diet [PURINA 5L79 from PMI Nutritional, USA]

Acclimatization: Animals have been acclimatized to the animal house environment for 2 weeks. The animal experiments were designed and conducted strictly in accordance with the guidelines of AEC. Streptozotocin (STZ) was freshly prepared in 0.1M citrate buffer (pH 4.5). T2D was induced experimentally by single intraperitoneal (i.p.) injection of low dose STZ (40 mg per kg) to the rats, kept fasted overnight. Animals were unfed for 12 h before the estimation of fasting blood glucose. When the fasting plasma glucose level had become above 250 mg dL-1 in 72 h after STZ administration, STZ treated animals were considered as diabetic and all the experimental animals were then kept without any treatment for a period of 2 weeks. The composition treatment was started on 15th day after STZ injection in the respective groups of rats.

### EXPERIMENTAL DESIGN

Test sample was dissolved in water containing 0.1% v/v Dimethyl Sulfoxide (DMSO) for animal treatment. Diabetic rats were then treated with intracerebroventricular (i.c.v.) administration of test samples. The animals were divided in six groups each of which contained six rats:

### Group, Designation and Dose Levels:

TABLE 1
Animal grouping and treatment details

| | | | No. of |
|---|---|---|---|
| Groups | Group Description | Treatment Description | animals |
| G1 | Normal control group | 0.1% v/v DMSO as vehicle by i.p., | 06 |
| | | route | |
| G2 | Diabetic control group | rats were made diabetic by a single | 06 |
| | | i.p., administration of low dose | |
| | | STZ, 40 mg kg-1 body weight) | |
| G3 | Diabetic group treated with | Equivalent human dose 100 mcg | 06 |
| | (2Z,4E)-5-[(1S)-1-hydroxy-2,6,6- | by i.p. route on every day for 28 | |
| | trimethyl-4-oxocyclohex-2-en-1- | days starting from day 15 after the | |
| | yl]-3-methylpenta-2,4-dienoic | STZ injection | |
| | acid | | |
| G4 | Diabetic group treated with 3- | Equivalent human dose 250 mg | 06 |
| | Aminoisobutyric acid | by i.p. route on every day for 28 | |
| | | days starting from day 15 after the | |
| | | STZ injection | |
| G5 | Diabetic group treated with1- | Equivalent human dose 35 mg | 06 |
| | isothiocyanato-4-methylsulfinyl | by i.p. route on every day for 28 | |
| | butane | days starting from day 15 after the | |
| | | STZ injection | |
| G6 | Diabetic group treated with | Equivalent human dose 100 mg | 06 |
| | Oxobutanedioic acid | by i.p. route on every day for 28 | |
| | | days starting from day 15 after the | |
| | | STZ injection | |
| G7 | Diabetic group treated with (2S)- | Equivalent human dose 250 mg | 06 |
| | 2-[[(2S)-2- | by i.p. route on every day for 28 | |
| | hydroxypropanoyl]amino]-3- | days starting from day 15 after the | |
| | phenylpropanoic acid | STZ injection | |
| G8 | Diabetic group treated with | Equivalent human dose 250 mg | 06 |
| | Pentadecanoic acid | by i.p. route on every day for 28 | |
| | | days starting from day 15 after the | |
| | | STZ injection | |
| G9 | Diabetic group treated with | Equivalent human dose 250 mg | 06 |
| | Dileucine | by i.p. route on every day for 28 | |
| | | days starting from day 15 after the | |
| | | STZ injection | |
| G10 | Diabetic group treated with | Equivalent human dose 10 mg | 06 |
| | (2R,4aS,6aR,6aS,14aS,14bR)-10- | by i.p. route on every day for 28 | |
| | hydroxy-2,4a,6a,6a,9,14a- | days starting from day 15 after the | |
| | hexamethyl-11-oxo- | STZ injection | |
| | 1,3,4,5,6,13,14,14b- | | |
| | octahydropicene-2-carboxylic | | |
| | acid | | |
| G11 | Diabetic group treated with | Composition 1 by i.p. route on | 06 |
| | Composition 1 | every day for 28 days starting from | |
| | | day 15 after the STZ injection | |
| G12 | Diabetic group treated with | Composition 2 by i.p. route on | 06 |
| | Composition 2 | every day for 28 days starting from | |
| | | day 15 after the STZ injection | |
| G13 | Diabetic group treated with | Composition 3 by i.p. route on | 06 |
| | Composition 3 | every day for 28 days starting from | |
| | | day 15 after the STZ injection | |
| G14 | Diabetic group treated with | Composition 4 by i.p. route on | 06 |
| | Composition 4 | every day for 28 days starting from | |
| | | day 15 after the STZ injection | |
| G15 | Diabetic group treated with | | |
| | Composition 5 | every day for 28 days starting from | |
| | | day 15 after the STZ injection | |
| G16 | Diabetic group treated with | Composition 6 by i.p. route on | 06 |
| | Composition 6 | every day for 28 days starting from | |
| | | day 15 after the STZ injection | |
| G17 | Diabetic group treated with | Composition 7 by i.p. route on | 06 |
| | Composition 7 | every day for 28 days starting from | |
| | | day 15 after the STZ injection | |

Daily food and water consumptions were observed and body weights were taken on a weekly basis. After 72 h of STZ administration, blood was drawn from tail vein of rats and fasting blood glucose was measured by glucometer. On the last day of the experiment, all rats were sacrificed by common method of euthanasia. All animals were unfed for 12 h before being killed. Liver, kidneys, pancreas tissues and skeleton muscles were removed surgically and stored at −80° C. in a deep freezer for western blot analysis.

### Western Blot Analysis

GLUT4 and CD38 expressions were evaluated by Western blot. For GLUT4 content analysis, membrane samples were prepared from skeletal muscles and for CD38 protein analysis, liver tissues were used. In Western blot analysis, 25 μg of membrane protein per sample was subjected to Sodium Dodecyl Sulfate (SDS) polyacrylamide gel electrophoresis in parallel with molecular weight markers on a 10% resolving gel. The separated proteins were then transferred to a nitrocellulose membrane in a semidry system. β-tubulin was used to ensure equivalent loading of the gel incubating the same membrane with β-tubulin antibody after stripping.

Western blotting kit was used for immunodetection, using anti-GLUT4 and anti-CD38 antibodies. Detection was done by the enhanced chemiluminescence method and densitometric quantitation was done by scanning of the autoradiographic GLUT4 and CD38 signals with the help of software. Densitometric scanning was indicated the (percentage of band intensity) of GLUT4 bands and CD38 bands of test samples treated diabetic rats and diabetic control rats. Data were expressed as percentage increase or decrease of intensity of band.

### Statistical Analysis

Each value represents mean±SD (n=6). The significance of in vivo data was analyzed by one-way Anova followed by Dunnet test. P<0.05 was considered as significant.

### Results:

| | Band | | | % Decrease |
|---|---|---|---|---|
| | Intensity of | % Increase of | Band Intensity | of CD38 |
| | GLUT4 (%) | Glut4 over | of CD38 | Disease |
| Groups | 54 kDa | Disease control | (%) 45 kDa | control |
| G1 | 100 | 0.0 | 100 | 0 |
| G2 | 80 | . . . | 112 | . . . |
| G3 | 110 | 37.5 | 95 | 15.2 |
| G4 | 104 | 30.0 | 99 | 11.6 |
| G5 | 95 | 18.8 | 102 | 8.9 |
| G6 | 102 | 27.5 | 101 | 9.8 |
| G7 | 106 | 32.5 | 96 | 14.3 |
| G8 | 100 | 25.0 | 103 | 8.0 |
| G9 | 98 | 22.5 | 99 | 11.6 |
| G10 | 95 | 18.8 | 101 | 9.8 |
| G11 | 150 | 87.5 | 62 | 44.6 |
| G12 | 153 | 91.3 | 58 | 48.2 |
| G13 | 147 | 83.8 | 71 | 36.6 |
| G14 | 144 | 80.0 | 63 | 43.8 |
| G15 | 141 | 76.3 | 72 | 35.7 |
| G16 | 138 | 72.5 | 70 | 37.5 |
| G17 | 135 | 68.8 | 78 | 30.4 |

### DISCUSSION

GLUT4 is insulin-sensitive glucose transporter, mainly expressed in skeletal muscles, adipocytes and cardiac muscles. It was observed that sesquiterpenoid and glucose uptake promoters combination facilitates GLUT4 translocation in skeletal muscles of test sample treated diabetic rats than that of the diabetic control rats. CD38 enzyme is a NAD+ase which degrades NAD+ in tissue that leads to metabolic syndrome. The test samples were downregulated CD38 expression in type II diabetic rats.

The present data showed prominent effect with the Group 3 combinations i.e. G11 to G17 where pharmacological downregulation or inhibition of CD38 expression synergistically work with upregulation or enhancement of Glut4 expression.

Based on the results obtained, it is concluded that, enhanced GLUT4 translocation of treated groups G3 to G17 indicate more glucose lowering as well as B-cell preserving efficacy, where the downregulation of CD38 with treated groups G3 to G17 revealed that alone G3 is not sufficient to give significant results. The combination groups (G11-G17) are more effective in improving glucose homeostasis and metabolic diseases such as T2D, obesity and obesity related diseases and protect from inevitably damages vital organs.

1. A synergistic bioactive composition for treating upregulated cyclic ADP ribose hydrolase (CD38) disorders, wherein the composition comprising exogenous combination of;
a) bioactive sesquiterpenoid;
b) at least one glucose uptake promoter;
c) at least one pharmaceutically acceptable excipients.

2. The composition as claimed in claim 1, wherein the bioactive sesquiterpenoid is present in the in the range of 0.001% to 1% by weight of the total composition.

3. The composition as claimed in claim 1, wherein the glucose uptake promoter is present in the in the range of 10% to 99% by weight of the total composition.

4. The composition as claimed in claim 1, wherein the bioactive sesquiterpenoid is (2Z,4E)-5-[(1S)-1-hydroxy-2,6,6-trimethyl-4-oxocyclohex-2-en-1-yl]-3-methylpenta-2,4-dienoic acid or its salts thereof.

5. The composition as claimed in claim 1, wherein the glucose uptake promoter(s) is selected from the group consisting of Oxobutanedioic acid; 2-Oxo-1,5-pentanedioic acid; (3β,5ß,7α,12α)-7,12-dihydroxy-3-[(1-oxoeicosyl)amino]-cholan-24-oic acid; (2R)-3-(Allylthio)-2-aminopropanoic acid; 1-(4-Aminobutyl) guanidine; dileucine; (3R)-3-acetyloxy-4-(trimethylazaniumyl) butanoate; 3-amino-2-methylpropanoic acid; (2S)-2-[[(2S)-2hydroxypropanoyl]amino]-3-phenylpropanoic acid, Pentadecanoic acid, (2R,3S,4S)-pentane-1,2,3,4,5-pentol, 1-isothiocyanato-4-methylsulfinylbutane, 3-Aminoisobutyric acid, (2R,4aS,6aR,6aS,14aS,14bR)-10-hydroxy-2,4a,6a,6a,9,14a-hexamethyl-11-oxo-1,3,4,5,6,13, 14,14b-octahydropicene-2-carboxylic acid or salts thereof.

6. The composition as claimed in claim 1, wherein the pharmaceutically acceptable excipients are selected from a group consisting of the diluent which is present in a range of 1 to 30%; the binder which is present in a range of 0.1 to 25%; the lubricant which is present in a range of 0.1 to 10.0%; the glidant which is present in a range of 0.1 to 5.0%; the additive which is present in a range of 0.1 to 10%; the surfactant which is present in a range of 0.1 to 5.0%; the stabilizer which is present in a range of 0.1 to 5.0%; %; the antioxidant which is present in a range of 0.1 to 5.0%; and the plasticizer which is present in a range of 0.1 to 5.0%; by weight of total composition.

7. The composition as claimed in claim 1, wherein the pharmaceutically acceptable excipient is further comprising the buffering or alkalizing agent is present in a range of 20% to 50% by weight of the total composition/formulation.

8. The composition as claimed in claim 1, wherein the composition downregulates CD38 expression by 30% to 48% and upregulates GLUT4 expression by 68% to 91% as compared with disease control.

9. The composition as claimed in claim 1, wherein the effective unit dose is formulated in solid form in the range of 10 to 1000 mg.

10. The composition as claimed in claim 1, wherein the upregulated cyclic ADP ribose hydrolase (CD38) disorders are selected from weight gain, obesity, adiposity, dyslipidemia, diabetes, prediabetes, liver diseases, hypertension, sexual dysfunction, insulin resistance, hyperglycemia, glucose intolerance, inflammation, polycystic ovary syndrome (PCOS), neurodegenerative diseases, cardiovascular diseases, osteoarthritis, musculoskeletal diseases, neuropathic pain and kidney diseases.
